(19) United States
(12) Patent Application Publication
(10) Pub. No.: US 2024/0058212 A1
(43) Pub. Date: Feb. 22, 2024

(54) METHODS FOR PREDICTING SAFETY AND EFFECTIVENESS OF SPINAL THERMAL DEVICES

(71) Applicants: CERAGEM Co., Ltd., Cheonan-si (KR); CERAGEM CLINICAL, INC., Seoul (KR)

(72) Inventor: Kiwon LEE, Hanam-si (KR)

(21) Appl. No.: 18/334,954

(22) Filed: Jun. 14, 2023

(30) Foreign Application Priority Data
Aug. 16, 2022 (KR) 10-2022-0102106

(51) Int. Cl. A61H 39/06; A61B 5/01; A61B 5/00

(52) U.S. Cl. CPC A61H 39/06; A61B 5/01; A61B 5/7275; A61H 2201/0173; A61H 2201/0207; A61H 2201/5007; A61H 2205/081

(57) ABSTRACT

The present disclosure relates to a method for predicting safety and effectiveness of a massage device, and more particularly to a method for predicting safety and effectiveness of a spinal thermal device. According to an aspect of the present disclosure, a method for predicting safety and effectiveness of the spinal thermal device may include generating three-dimensional structure data of a user's body, generating three-dimensional structure data of the spinal thermal device, setting a set value of the spinal thermal device, calculating thermal conduction and temperature change of the user's body due to heat applied in a process of pressurizing the user's body as the spinal thermal device operates as the set value, converting the temperature change value of the user's body into a blood circulation value, and visualizing the blood circulation value of the user's body.

### CROSS-REFERENCE TO RELATED APPLICATION

This application claims priority to and the benefit of Korean Patent Application No. 10-2022-0102106, filed on Aug. 16, 2022, the disclosure of which is incorporated herein by reference in its entirety.

The disclosure relates to a method for predicting safety and effectiveness of a massage device, and more particularly, to a method for predicting safety and effectiveness of a spinal thermal device.

### BACKGROUND

Spinal thermal devices are widely used to alleviate acute or chronic pain generated in muscles and nerve tissues of spinal region caused by continuing to work in an inappropriate posture for a long time or by habituation of these postures, and to move along a body part for the purpose of improving the blood circulation of the body or relieve instantaneous muscle stiffness, and to improve blood flow by applying heat stimulation to the site of pain.

### SUMMARY

Aspects of the disclosure provide a method for predicting safety and effectiveness of a spinal thermal device, which can optimize an effect of improving blood flow provided to a targeted region using the spinal thermal device.

The disclosure is not limited to the above-described tasks, and other tasks not described herein will be clearly understood by those skilled in the art from the following description.

According to an aspect of the disclosure, there is provided a method of predicting safety and effectiveness of a spinal thermal device, the method including: a step of generating three-dimensional structure data of a user's body; a step of generating three-dimensional structure data of the spinal thermal device; a step of setting a set value of the spinal thermal device; a step of calculating a thermal conduction and a temperature change of the user's body due to heat applied in a process of pressurizing the user's body as the spinal thermal device operates as the set value; a step of converting the temperature change value of the user's body into a blood circulation value; and a step of visualizing the blood circulation value of the user's body.

In this case, the calculating of the thermal conduction and the temperature change of the user's body due to heat applied in a process of pressurizing the user's body may include: visualizing the thermal conduction and the temperature change value of the user's body.

In this case, the calculating the thermal conduction and the temperature change due to a warming effect during the user's body pressurization may be: calculating a temperature change of a portion corresponding to a depth of 3 cm from a skin surface of the user during a process of pressurizing the user's body while a ceramic of the spinal thermal device rises along a height direction.

In this case, the calculating the thermal conduction and the temperature change of the user's body may be: calculating the thermal conduction and the temperature change through Equation 1 below:

$$ρ c \frac{\partial T}{\partial t} = ∇ \cdot ( k ∇ T ) + {ρ}_{b} {c}_{b} {w}_{b} ( T - {T}_{b} ) + {Q}_{m} \qquad ( 1 )$$

where ρ is tissue density (units of kg/m³), c is specific heat capacity (J kg⁻¹ K⁻¹), T is temperature (K), t denotes time (s), k is thermal conductivity (W m⁻¹ K⁻¹), ρ_{b} is density of blood (kg m⁻³), c_{b} is specific heat capacity of blood (J kg⁻¹ K⁻¹), w_{b} is perfusion rate of blood (1/s), T_{b} is blood temperature (K), Qₘ is rate of metabolic heat generation (W/m³), and solution is carried out under steady state conditions.

In this case, the calculating through the Equation 1 may further include: calculating a convective heat loss from boundaries of the user's body to external environment through Equation 2 below:

q₀=h(T_{amb}−T) (2)
where q₀ (W m⁻²) is convective heat flux, h is heating transfer coefficient for the tissues comprising the model (e.g., user's body), and is 5 W m⁻² K, T_{amb} is ambient temperature (e.g., 25° C.), initial temperature of the tissues is set to 37° C., temperature of the ceramic is fixed within a simulation to a value ranging from 45 to 65° C., and external boundary of a skin in contact with a bed surface, corresponding to a heating mat, is assigned a temperature of 40° C.

In this case, the converting the temperature change value of the user's body into the blood circulation value may be: converting the temperature change value of the user's body into a blood circulation value through Equation 3 below:

F(ΔT)=F₀(Fₘₐₓ−Fₒ)e^{-(aΔT+b)} (3)
where F is blood flow (L/min), ΔT is change in temperature at the site of vessel (° C.), Fₒ is blood flow in absence of exogenous heating, Fₘₐₓ is maximum achievable blood flow due to physical limitations of cardiovascular system, and the free model parameters a and b are estimated by fitting the user's body to empirical measurements reported by Chiesa and colleagues.

In this case, the setting the set value of the spinal thermal device may be: setting a ceramic temperature, a ceramic height, and a heating element temperature.

In this case, the method may further include: after the visualizing of the blood circulation value of the user's body, deriving a set value for obtaining an optimal temperature or an optimal blood circulation value of the user's body within a range of a set ceramic temperature, a ceramic height, and a heating element temperature.

In this case, the deriving the set value for obtaining an optimal temperature or an optimal blood circulation value of the user's body may be: deriving the set value for obtaining the optimal temperature or the optimal blood circulation value for each user's body type according to a change in structure according to the user's body type.

In this case, the converting the temperature change value of the user's body into the blood circulation value may further include: correcting the temperature change value of the user's body by reflecting blood circulation data for each user's body position.

In this case, the correcting the temperature change value of the user's body may further include: analyzing a laser received from the blood of the user.

In this case, the three-dimensional structure data of the user's body may be three-dimensional structure data classified into skin, subcutaneous fat, soft tissue, muscles, vertebrae, intervertebral disc, epidural fat, cerebrospinal fluid, and spinal cord.

According to the above configuration, the method for predicting the safety and effectiveness of the spinal thermal device according to the embodiment of the disclosure generates three-dimensional structure data of the user's body and three-dimensional structure data of the spinal thermal device, calculates thermal conduction and temperature change of the user's body due to the heat while the spinal thermal device operates according to the set value, and then converts the thermal conduction and temperature change into a blood circulation value, thereby optimizing the effect of improving the blood flow provided to the targeted region, through this the safety and effectiveness of the spine thermal device can be predicted.

It should be understood that the effects of the disclosure are not limited to the above-described effects, and include all effects that can be inferred from the configuration of the disclosure described in the detailed description or claims of the disclosure.

### BRIEF DESCRIPTION OF THE DRAWINGS

FIG. 1 is a flowchart illustrating a method for predicting safety and effectiveness of a spinal thermal device according to an example embodiment of the present disclosure.

FIG. 2 is a perspective view illustrating a spinal thermal device according to an example embodiment of the present disclosure.

FIG. 3 is a plan view illustrating a spinal thermal device according to an example embodiment of the present disclosure.

FIGS. 4 to 7 are computer models constructed to predict tissue temperature and blood flow during thermal massage using a spinal thermal device according to an example embodiment of the present disclosure,

FIG. 4 is a view illustrating a sagittal slice of T2-weighted anatomical MRI used to construct a computational model developed in the present disclosure,

FIG. 5 is a view illustrating physical properties of governing physiological response to an external heat source after segmenting a volume into nine tissues based on image contrast,

FIG. 6 is a view illustrating a result of yielding the temperature during thermal massage throughout the volume by solving Pennes bio-heat equation with a finite element model (FEM) solver, where positions of four ceramics can be confirmed from temperatures hot-spot in a superficial slice, and

FIG. 7 is a view illustrating an empirically-guided sigmoidal model for translating tissue temperature to the predicted changes in blood flow.

FIG. 8 is a view illustrating quantifying temperature increase of the part in contact with ceramic during thermal massage using a spine thermal device according to an embodiment of the present disclosure, (A) is a diagram illustrating a temperature of a sagittal slice of the volume during thermal massage with the ceramic set to 65° C., (B) is a diagram illustrating contours of the temperature distribution shown in (A).

FIG. 9 is a view illustrating quantifying temperature increase between ceramics adjacent to each other disposed along a width direction during thermal massage using a spine thermal device according to an embodiment of the present disclosure, (A) is a diagram illustrating a temperature of a sagittal slice, (B) is a diagram illustrating contours of the temperature distribution shown in (A).

FIG. 10 is a view illustrating a temperature distribution for each body depth of a user according to a ceramic temperature of a spine thermal device according to an embodiment of the present disclosure, (A) is a diagram illustrating a temperature distribution of the part in contact with ceramic during thermal massage and (B) is a diagram illustrating a temperature distribution between ceramics adjacent to each other disposed along a width direction.

FIG. 11 is a view illustrating a temperature distribution for each body width direction position of a user according to a ceramic temperature of the spine thermal device according to an embodiment of the present disclosure, (A) is a diagram illustrating a temperature distribution for each body width direction position at a depth of 2 cm of the user's body, and (B) is a diagram illustrating a temperature distribution for each body width direction position at a depth of 3 cm of the user's body.

FIG. 12 is a view illustrating a quantification of blood flow distribution of a portion in contact with ceramics during thermal massage using the spinal thermal device according to an embodiment of the present disclosure, (A) shows a blood flow distribution (pronounced increases in blood flow are observed at greater depths compared to a temperature distribution) during thermal massage with the ceramics set at 65° C., (B) shows contours of the blood flow distribution indicated in (A) (the concentration of the contours indicates a transition region from a high blood flow to a low blood flow, and the transition region is identified at a deep depth).

FIG. 13 is a view illustrating a quantification of blood flow distribution between the ceramics adjacent to each other along a width direction during thermal massage using the spinal thermal device according to an embodiment of the present disclosure, (A) shows a blood flow distribution of a sagittal slice (the increase in blood flow is visibly dampened), (B) shows contours of the blood flow distribution indicated in (A) (a transition region is observed at a relatively shallow depth).

FIG. 14 is a view illustrating a blood flow distribution according to a body depth of a user according to the ceramics temperature of the spinal thermal device according to an embodiment of the present disclosure, (A) shows a blood flow distribution of a portion in contact with the ceramics (due to a sigmoidal nature of a temperature-circulation relationship, a blood flow falloff with depth occurs at a larger depth between 2-4 cm, depending on a set value, and four-fold increases in circulation are predicted at depths of 2-3 cm), (B) shows a blood flow distribution between the ceramics adjacent to each other along a width direction (the increase in blood flow is no longer apparent beyond a depth of 3-4 cm).

FIG. 15 is a view illustrating a blood flow distribution for each body width direction position of a user according to the ceramics temperature of the spinal thermal device according to an embodiment of the present disclosure, (A) shows a blood flow distribution according to the body width direction position based on a depth of 2 cm, and (B) shows a blood flow distribution according to the body width direction position based on a depth of 3 cm (the blood flow reaches a three-fold increase at the ceramics temperature of 55° C.).

### DETAILED DESCRIPTION

Hereinafter, embodiments of the present disclosure will be described in detail with reference to the accompanying drawings so that those skilled in the art can easily carry out the present disclosure. The present disclosure may be embodied in various different forms and is not limited to the embodiments described herein. In order to clearly describe the present disclosure, parts not related to the description are omitted in the drawings, and the same or similar components are denoted by the same reference numerals throughout the specification.

The words and terms used in the specification and the claims are not interpreted as limited to ordinary or dictionary meanings, but should be interpreted as meanings and concepts corresponding to technical aspects of the present disclosure according to principles capable of defining terms and concepts by the inventor in order to describe the disclosure in the best way.

In order to provide an optimal thermal effect using a spinal thermal device, quantitative information on how much a temperature of a ceramic required to improve blood flow of a targeted region (e.g., a specified muscle group) is needed.

However, spinal heating devices according to one example technology do not consider the targeted region, heat the ceramic at the temperature selected by the user, and then perform the thermal massage, so the blood flow improvement effect of the targeted region is not constant, and if the temperature of the ceramic is excessively increased to enhance the effect of improving blood flow, there is a risk that the user may get burned.

Therefore, there is a need to develop a technology capable of optimizing the effect of improving blood flow provided through the spinal thermal device.

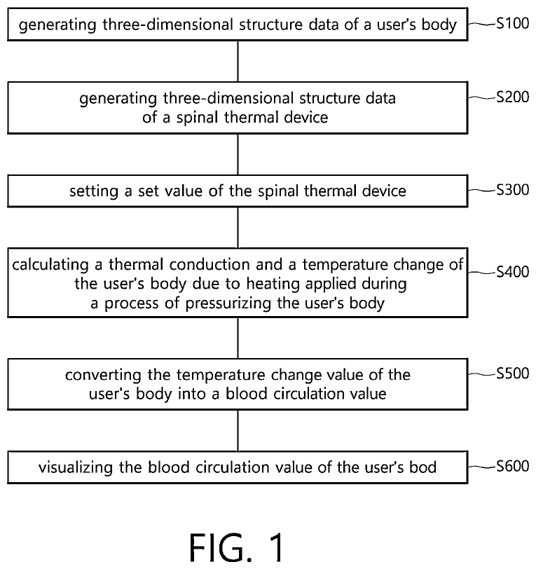

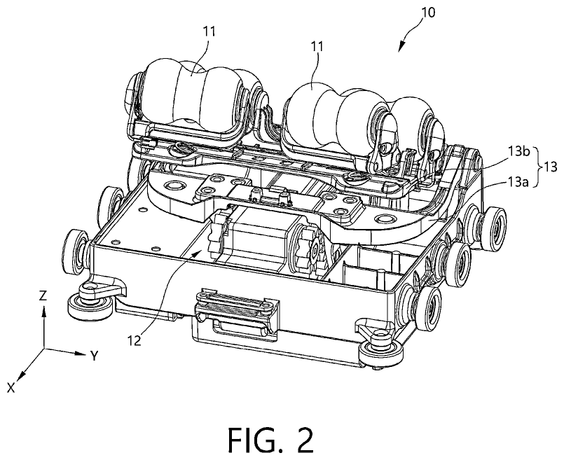

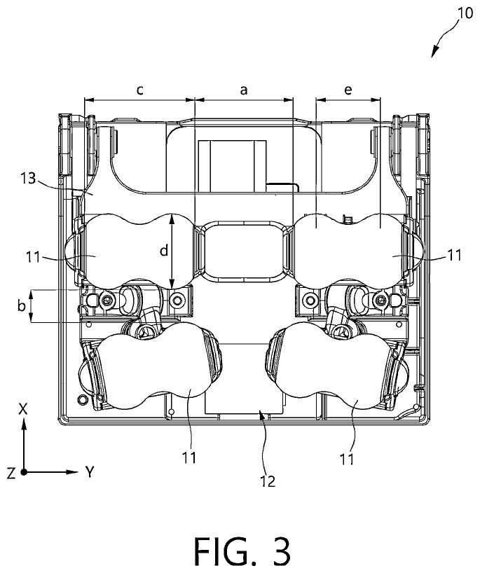

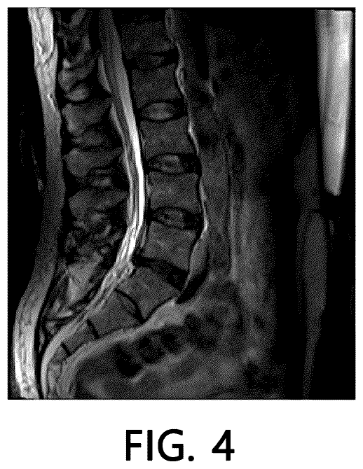

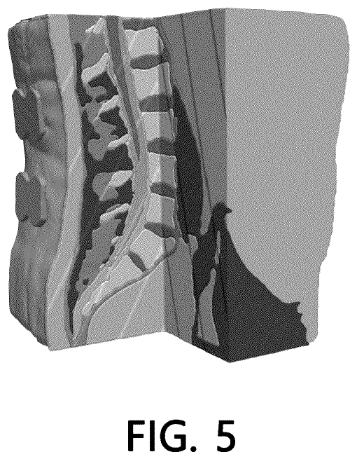

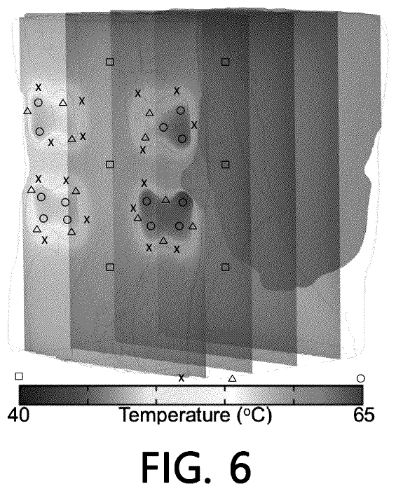

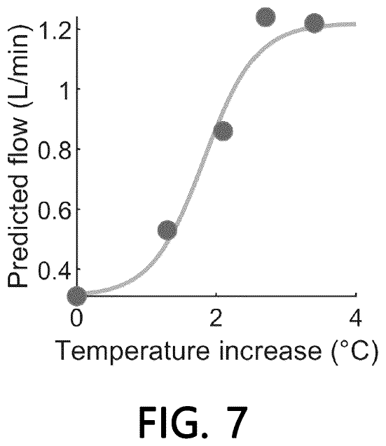

FIG. 1 is a flowchart illustrating a method for predicting safety and effectiveness of a spinal thermal device according to an embodiment of the present disclosure, FIG. 2 is a perspective view illustrating a spinal thermal device according to an embodiment of the present disclosure, FIG. 3 is a plan view illustrating a spinal thermal device according to an embodiment of the present disclosure, FIGS. 4 to 7 are computer models constructed to predict tissue temperature and blood flow during thermal massage using a spinal thermal device according to an embodiment of the present disclosure, FIG. 4 is a view illustrating a sagittal slice of T2-weighted anatomical MRI used to construct a computational model developed in the present disclosure, FIG. 5 is a view illustrating physical properties of governing physiological response to an external heat source after segmenting a volume into nine tissues based on image contrast, FIG. 6 is a view illustrating a result of yielding the temperature during thermal massage throughout the volume by solving Pennes bio-heat equation with a finite element model (FEM) solver, where positions of four ceramics can be confirmed from temperatures hot-spot in a superficial slice, and FIG. 7 is a view illustrating an empirically-guided sigmoidal model for translating tissue temperature to the predicted changes in blood flow. At this time, the X axis means a direction in which the spinal thermal device moves along a user's body to thermally massage the user in a longitudinal direction (axial direction), the Y axis means a direction in which the spinal thermal device moves along a lateral direction of the user in a width direction, and the Z axis means a direction in which the spinal thermal device moves upward to press the user in a height direction. In order to clearly describe the present disclosure, parts not related to the description are omitted in the drawings.

As shown in FIG. 1, according to an embodiment of the present disclosure, there is provided a method for predicting safety and effectiveness of a spinal thermal device, the method comprising generating three-dimensional structure data of a user's body using a computer (Step 100), generating three-dimensional structure data of the spinal thermal device 10 (Step 200), setting a set value of the spinal thermal device 10 (Step 300), calculating a thermal conduction and a temperature change of the user's body due to heat applied in a process of pressurizing the user's body as the spinal thermal device 10 operates as the set value (Step 400), converting the temperature change value of the user's body into a blood circulation value (Step 500), and visualizing the blood circulation value of the user's body (Step 600).

At this time, the computer may include a notebook, a laptop, a tablet PC, a slate PC, and the like equipped with a web browser (WEB Browser), and the computer may comprise a controller for performing each of the above steps, an input unit for inputting a user command, and an output unit for showing a resultant to the user.

The controller may include a processor, a ROM in which a control program is stored, and a RAM that stores an input signal or data or is used as a storage area corresponding to various operations to be performed.

Also, a graphic processing board including a processor, a RAM, or a ROM may be included in a separate circuit board electrically connected to the controller. The processor, the RAM, and the ROM may be connected to each other through an internal bus.

Also, the controller may be used as a term referring to a component including the processor, the RAM, and the ROM. Also, the controller may be used as a term referring to a component including the processor, the RAM, the ROM, and the processing board.

The input unit receives a predetermined instruction or command from a user for controlling the computer. For example, the input unit may include a user interface such as a keyboard, a mouse, a trackball, a time gain compensation (TGC) control knob, a lateral gain compensation (LGC) control knob, or a paddle.

The output unit may display a resultant on one or more display areas. The user may check the visualized result through the display area.

In this case, data related to the user's body may be pre-held user's body data, and may be computed tomography (CT) or magnetic resonance imaging (MRI) data. Also, the user's body data may be three-dimensional structure data classified into skin, subcutaneous fat, soft tissue, muscles, vertebrae, intervertebral disc, epidural fat, cerebrospinal fluid, and spinal cord. When the user's body data is modeled and simulated as described above, the effect of improving blood flow provided to a target site may be optimized, and thus safety and effectiveness of the spinal thermal device may be predicted.

A T2-weighted MRI of a healthy male adult (BMI: 25 kg/m², age: 42 years) was performed by using a 3 T Siemens MAGNETOM Prisma scanner equipped with a CP Spine array coil (Siemens Healthineers, PA, USA), and FIG. 4 shows a sagittal slice of the user. Parameters of image acquisition was set according to: TE=99 ms, TR=7,040 ms, flip angle=130°, FOV=256 mm, SNR=1, in-plane resolution=256×256, slice thickness=1 mm, and voxel size: 1×1×1 mm.

Based on image intensity, the MRI was segmented into nine tissue masks: skin, subcutaneous fat, soft tissue, muscles, intervertebral disc, vertebrae, epidural fat, cerebrospinal fluid, and spinal cord. Manual tissue segmentation was carried out by first thresholding the images, followed by morphological filtering such as flood fill, dilation, and erosion, performed in Simpleware ScanIP (Synopsys Inc., CA, USA). To ensure tissue continuity and improve segmentation accuracy, the data was extensively visualized and compared with the anatomy of the subject's spine while performing manual adjustments to the tissue masks. Thicknesses of the tissues comprising the imaged region were measured as: (skin) 1.1 mm, (subcutaneous fat) 13 mm, (muscle) 42 mm, (soft tissue) 2.8 mm, (epidural fat) 2.2 mm, (CSF) 2.9 mm, and (spinal cord) 1.9 mm. Thus, the muscle (e.g., the presumed target of thermal massage) occupied a region approximately 1.5-5.5 cm from the surface.

Regarding the generation of the three-dimensional structure data of the spinal thermal device 10, the tissue masks generated by the segmentation process were utilized to construct a finite element model (FEM) of the lumbar back. To this end, FEM consisting of more than 3.38 million tetrahedral elements was generated using Simpleware Scan IP software running tetrahedral voxel-based meshing algorithm. The resulting model was then integrated with separate domains that captured the position, orientation, and geometry of a thermal massage device. For example, the spinal thermal device 10 may model the characteristics of a CGM MB-1901 massage bed (CERAGEM Co. Ltd., Cheonan, Korea). The massage bed has two types of heat sources. The bed surface provides a diffuse layer of heat and four ceramics 11 that make direct contact with the body to provide more punctate and intense thermal stimulation. The spinal thermal device 10 includes four ceramics 11 arranged as the vertices of a rectangle. A width direction Y distance (a) between the ceramics 11 is 56 mm, and a length direction X distance between top and bottom ceramics 11 is 32 mm. Specifically, in Solidworks (Dassault Systems, MA, USA), the cross-section of the ceramics 11 was modeled by having a width (c) of 65 mm for the four ceramics 11, a diameter of the circular ends (d) of 45 mm, and a spacing (e) between the two circular ends is 30 mm, and the components were imported into Simpleware ScanIP for positioning and meshing.

The spinal thermal device 10 may include a driving unit 12 for adjusting the position of the ceramics 11 in the length direction X, and a support unit 13 for supporting the ceramics 11. The support unit 13 may include a first support member 13a for supporting the ceramics 11 disposed on one side of the length direction X and a second support member 13b for supporting the ceramics 11 disposed on the other side of the length direction X.

After generating the three-dimensional structure data of the spinal thermal device 10 as described above, the set value of the spinal thermal device 10 is set. The set value may be a temperature of the ceramics 11 according to the thermal massage mode selected by the user, a height of the ceramics 11, a temperature of the heating element, and the like.

Thereafter, as the set value, as the spinal thermal device 10 operates, the thermal conduction and temperature change of the user's body due to heat applied in a process of pressing of the user's body are calculated. In this case, the thermal conduction and temperature change of the skin, muscle, vertebrae, intervertebral disc of the user may be calculated.

After generating the 3D structure data of the spinal thermal device 10, the resulting model was imported into COMSOL Multiphysics 5.5 (COMSOL Inc., MA, USA), and then the Pennes' bio-heat transfer equation for governing heating during the thermal was solved as shown in Equation 1 below. Through this, the thermal conduction and temperature change of the user's body may be calculated.

$$ρ c \frac{\partial T}{\partial t} = ∇ \cdot ( k ∇ T ) + {ρ}_{b} {c}_{b} {w}_{b} ( T - {T}_{b} ) + {Q}_{m} \qquad ( 1 )$$

At this time, ρ is tissue density (units of kg/m³), c is specific heat capacity (J kg⁻¹ K⁻¹), T is temperature (K), t denotes time (s), k is thermal conductivity (W m⁻¹ K⁻¹), ρ_{b} is density of blood (kg m⁻³), c_{b} is specific heat capacity of blood (J kg⁻¹ K⁻¹), w_{b} is perfusion rate of blood (1/s), T_{b} is blood temperature (K), Qₘ is rate of metabolic heat generation (W/m³). The solution is carried out under steady state conditions.

That is, the thermal conduction and temperature change due to heat applied while pressing the user's body are calculated, and it is possible to check whether there is safety when the presses and heating are applied to the user's body.

The physical and thermal properties of the user's body tissue were assigned according to the values listed in Table 1 below (the thermal properties of the tissue and source comprising the computational model to estimate the temperature gradient during the thermal massage).

TABLE 1

| Material | [symbol] | [symbol] | c | [symbol] | [symbol] | [symbol] | [symbol] | [symbol] |
|---|---|---|---|---|---|---|---|---|
| Units | Wm⁻¹ K⁻¹ | kg m⁻³ | [symbol] | kg m⁻³ | [symbol] | [symbol] | K | W/m² |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | 0 | 0 | 0 | [symbol] | 0 |
| [symbol] | [symbol] | [symbol] | [symbol] | [symbol] | [symbol] | [symbol] | [symbol] | [symbol] |
| [symbol] | [symbol] | [symbol] | [symbol] | 0 | 0 | 0 | [symbol] | 0 |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |

[symbol]

indicates data missing or illegible when filed

In order to solve the bio-thermal equation (Equation 1), it is assumed that the temperature at external boundaries of the user's body was fixed to core body temperature (37° C.), and the absence of convective gradients across all internal tissue boundaries. The convective heat loss from the model boundary (user's body boundary) to the environment was modeled as shown in Equation 2 below.

q₀=h(T_{amb}−T) (2)

At this time, q₀ (units of W m⁻²) is convective heat flux, h=5 W m⁻² K is heat transfer coefficient for the tissues comprising the model (e.g., user's body), T_{amb} is ambient temperature (e.g., 25° C.). Initial temperature of the tissues is set to 37° C. Temperature of the ceramic is fixed within a simulation to a value ranging from 45 to 65° C. External boundary of a skin in contact with a bed surface, corresponding to a heating mat, is assigned a temperature of 40° C.

A non-linear model for mapping in situ tissue temperature to the resulting blood flow was developed in order to estimate magnitude and distribution of the blood flow produced during the thermal massage. This model was guided by simultaneous empirical measurement of the temperature and blood flow in the user's leg during heat stress as conducted by Chiesa and colleagues. The general form of the model developed here is as follows and through this, the temperature change value of the user's body can be converted into the blood circulation value:

F(ΔT)=Fₒ+(Fₘₐₓ−Fₒ)e^{-(aΔT+b)} (3)

At this time, where Fₒ is blood flow (L/min), ΔT is change in temperature at the site of vessel (° C.), Fₒ is blood flow in absence of exogenous heating, Fmax is maximum achievable blood flow due to physical limitations of cardiovascular system, and the free model parameters a and b are estimated by fitting the model (e.g., user's body) to empirical measurements reported by Chiesa and colleagues.

Namely, the following (T, F) pairs of measurements were reported. Here, T was taken to be the temperature in the muscle of the leg (e.g., the deepest measurement taken and the one closest to the site of the vessel) and F was taken to be the empirical blood flow in the central femoral artery (CFA): (34.9° C., 0.31 L/min), (36.2° C., 0.53 L/min), (37.0° C., 0.86 L/min), (37.6° C., 1.24 L/min), and (38.3° C., 1.22 L/min).

From these empirical measurements, the values Fₒ=0.31 L/min and Fₘₐₓ=1.22 L/min were assigned to the baseline and maximum blood flow, respectively. The conventional least-squares fitting was then performed employing the Matlab (Mathworks, MA, USA) function Isqcurvefit. As shown in FIG. 7, the minimization procedure calculated values of a=−2.67 and b=4.88, producing excellent fits to the empirical measurements while capturing a sigmoidal transition from baseline to maximal flow. The resulting model was then employed to transform local temperature to the corresponding blood flow.

As shown in FIGS. 5 and 6, the contact heating was delivered through a contoured mat at 40° C. covering the full extent of the lower back, and four ceramics with a diameter of 4.5 cm and a height of 1 cm, arranged on the back as the vertices of a rectangle. The ceramics 11 temperature was varied from 45 to 65° C. in increments of 5° C. For each intensity, the temperature distribution throughout the lumbar region was numerically computed. The resulting results were then coupled to the predicted change in blood flow. This allows the increase in circulation as a function of the tissue depth and the conductor 11 temperature.

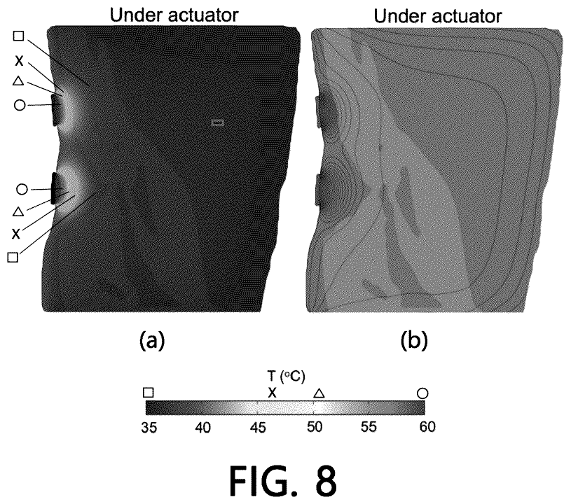

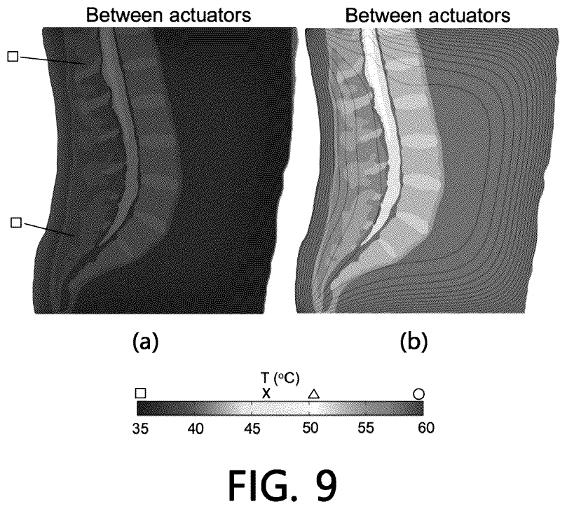

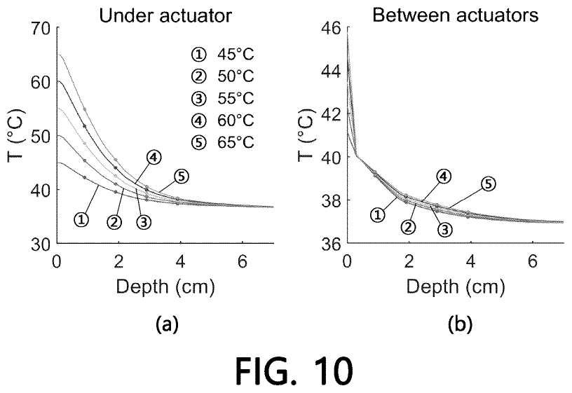

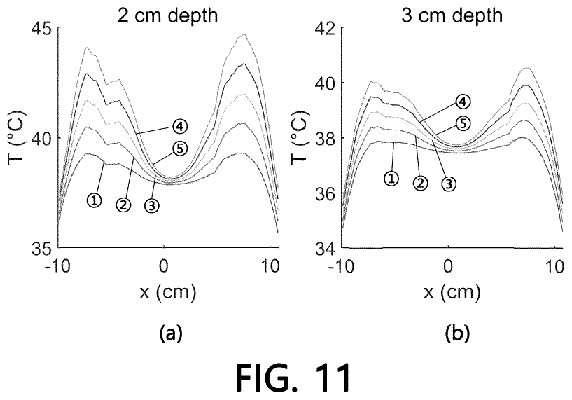

FIG. 8 is a diagram illustrating quantifying temperature increase of the part in contact with ceramic during thermal massage using a spine thermal device according to an embodiment of the present disclosure, (A) is a diagram illustrating a temperature of a sagittal slice of the volume during thermal massage with the ceramic set to 65° C., (B) is a diagram illustrating contours of the temperature distribution shown in (A), FIG. 9 is a diagram illustrating quantifying temperature increase between ceramics adjacent to each other disposed along a width direction during thermal massage using a spine thermal device according to an embodiment of the present disclosure, (A) is a diagram illustrating a temperature of a sagittal slice, (B) is a diagram illustrating contours of the temperature distribution shown in (A), FIG. 10 is a diagram illustrating a temperature distribution for each body depth of a user according to a ceramic temperature of a spine thermal device according to an embodiment of the present disclosure, (A) is a diagram illustrating a temperature distribution of the part in contact with ceramic during thermal massage and (B) is a diagram illustrating a temperature distribution between ceramics adjacent to each other disposed along a width direction, and FIG. 11 is a diagram illustrating a temperature distribution for each body width direction position of a user according to a ceramic temperature of the spine thermal device according to an embodiment of the present disclosure, (A) is a diagram illustrating a temperature distribution for each body width direction position at a depth of 2 cm of the user's body, and (B) is a diagram illustrating a temperature distribution for each body width direction position at a depth of 3 cm of the user's body.

The spatial distribution of temperature at the sagittal slice of the portion where the pair of ceramics 11 contacts with the temperature set to 65° C. is as shown in FIG. 8(A), and the contours of temperature distribution is as shown in FIG. 8(B). That is, the spatial dynamics of the temperature increase is apparent, and it can be seen that the largest change occurs near the stimulation site when considering that density of contour lines is higher.

The temperature immediately anterior to the centroid of the ceramics 11 was analyzed as a function of depth to quantify the effect of tissue depth on the ceramics 11 temperature. As shown in FIG. 10, the tissue temperature was found to be exhibited an exponential decay with depth (colors indicate the ceramic temperature, and markers indicate depths of 1, 2, 3, and 4 cm). As shown in FIG. 10(A), With the ceramics 11 temperature of 45° C., the temperature at depths of 1, 2, and 3 cm into the tissue was 42.2, 39.6, and 38.0° C., respectively. With an increased ceramics 11 temperature of 65° C., the corresponding temperatures were 54.9, 45.5, and 40.6° C. Therefore, the temperature gradient at the depth of 2 cm is in the range of 3-8° C., and the temperature gradient at the depth of 3 cm is 1-3° C.

Next, the effect of passive heating on tissue laterally displaced along the width direction Y at the portion in contact with the ceramics 11 was considered. FIG. 9 depicts the spatial distribution at a sagittal slice positioned at the midline (e.g., between pairs of ceramics, 6 cm from the ceramics centroid and 3.75 cm from the most lateral edge). As shown in FIG. 10(B), at the ceramics 11 temperature of 45° C., tissue temperatures were computed as 39.1° C., 37.9° C., and 37.5° C. at depths of 1, 2, and 3 cm, respectively. When a temperature of the ceramics 11 was 65° C., the temperature was only marginally higher than 39.3° C., 38.3° C., and 37.8° C., indicating that the temperature of the ceramics 11 did not transfer heat laterally along the width direction Y even if the temperature was increased.

The temperature was analyzed across the full lateral extent of the back to better characterize the effect of the thermal massage on the tissue temperature, but at fixed depth of the user's body was 2 cm (FIG. 11(A)) and 3 cm (FIG. 11(B)).

A vertical position in the height direction Z matching the pair of ceramics 11 arranged along the width direction Y was considered. The resulting temperature profile confirmed the local action of the ceramics 11, as the temperature fell to near mean body temperatures at the midline (37.9° C. at a depth of 2 cm as shown in FIG. 11(A), and 37.5° C. at a depth of 3 cm as shown in FIG. 11(B)).

As shown in the blue solid line of FIG. 11(A), with the ceramics 11 temperature of 45° C. evaluated at a depth of 2 cm, the temperature fell from a peak of 39.3, to 38.9° C. with a lateral displacement of 1 cm toward the midline, to 37.9° C. with a displacement of 2 cm, and then dropped to 36.4° C. with a displacement of 3 cm.

As shown in the green solid line of FIG. 11(A), With an elevated ceramics 11 temperature of 65° C., the corresponding values were 44.6° C. (peak), 43.8° C. (1 cm displacement), 41.6° C. (2 cm displacement), and 38.3° C. (3 cm displacement). As shown in FIG. 11(B), evaluated at a depth of 3 cm, the peak temperature with the ceramics 11 set to 45° C. was 38.0° C., which fell to 37.7° C., 37.1° C., and 35.2° C. when laterally displaced by 1, 2, and 3 cm, respectively. When simulating the ceramics 11 temperature of 65° C., the peak temperature of 40.5° C. dropped to 40.2° C., 39.2° C. and 36.7° C., when laterally displaced by 1, 2, and 3 cm respectively,

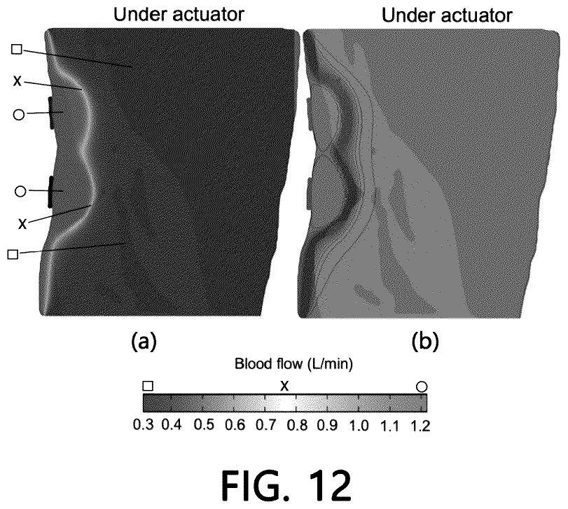

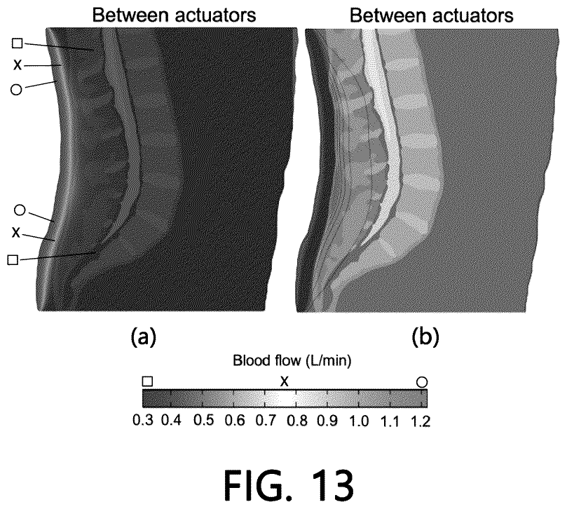

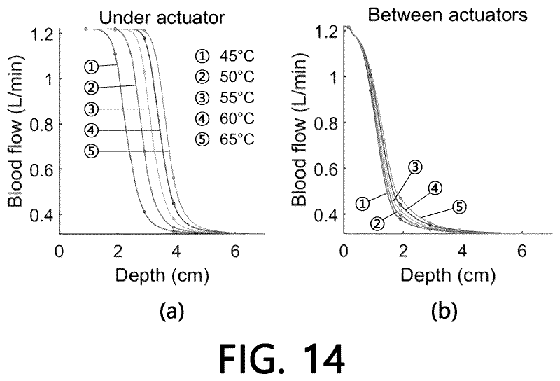

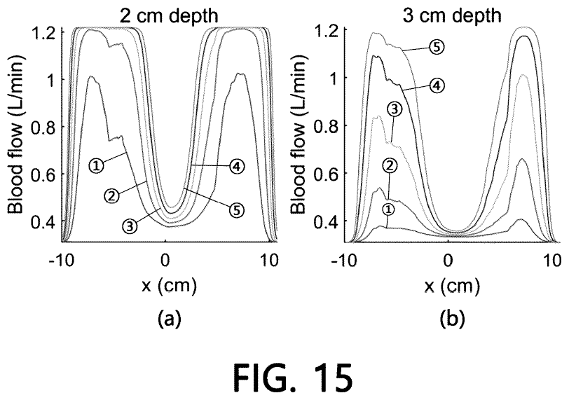

FIG. 12 is a diagram illustrating a quantification of blood flow distribution of a portion in contact with ceramics during thermal massage using the spinal thermal device according to an embodiment of the present disclosure, (A) shows a blood flow distribution (pronounced increases in blood flow are observed at greater depths compared to a temperature distribution) during thermal massage with the ceramics set at 65° C., (B) shows contours of the blood flow distribution indicated in (A) (the concentration of the contours indicates a transition region from a high blood flow to a low blood flow, and the transition region is identified at a deep depth), FIG. 13 is a diagram illustrating a quantification of blood flow distribution between the ceramics adjacent to each other along a width direction during thermal massage using the spinal thermal device according to an embodiment of the present disclosure, (A) shows a blood flow distribution of a sagittal slice (the increase in blood flow is visibly dampened), (B) shows contours of the blood flow distribution indicated in (A) (a transition region is observed at a relatively shallow depth), FIG. 14 is a diagram illustrating a blood flow distribution according to a body depth of a user according to the ceramics temperature of the spinal thermal device according to an embodiment of the present disclosure, (A) shows a blood flow distribution of a portion in contact with the ceramics (due to a sigmoidal nature of a temperature-circulation relationship, a blood flow falloff with depth occurs at a larger depth between 2-4 cm, depending on a set value, and four-fold increases in circulation are predicted at depths of 2-3 cm), (B) shows a blood flow distribution between the ceramics adjacent to each other along a width direction (the increase in blood flow is no longer apparent beyond a depth of 3-4 cm), and FIG. 15 is a diagram illustrating a blood flow distribution for each body width direction position of a user according to the ceramics temperature of the spinal thermal device according to an embodiment of the present disclosure, (A) shows a blood flow distribution according to the body width direction position based on a depth of 2 cm, and (B) shows a blood flow distribution according to the body width direction position based on a depth of 3 cm (the blood flow reaches a three-fold increase at the ceramics temperature of 55° C.).

In order to predict the magnitude of increased blood flow during thermal massage, a simple non-linear model of the relationship between in situ tissue temperature and blood flow was developed. The model was based on concurrent empirical measurements of temperature and blood flow in the user's leg during heat stress, where flow was found to non-linearly increase from a baseline value of 0.31 L/min to a maximum value of 1.22 L/min. The tissue temperature value computationally derived employing the resulting sigmoid model was as shown in FIGS. 8 to 11, and this was converted into a blood flow estimation value of the low back during thermal massage.

When the ceramics 11 temperature is 65° C., the spatial distribution of predicted blood flow is shown in FIGS. 12(A) and 12(B) for a sagittal slice of a portion of in contact with ceramics 11, and the spatial distribution of predicted blood flow of a mid-sagittal slice between the ceramics 11 adjacent to each other along a width direction Y is shown in FIGS. 13(A) and 13(B). That is, it is evident that blood flow exhibits a more favorable decay with depth.

As shown in blue solid line of FIG. 14(A), when the ceramics 11 temperature was 45° C., the predicted blood flow was 1.22 L/min, 1.11 L/min, 0.41 L/min, and 0.33 L/min at depths of 1, 2, 3, and 4 cm, respectively. As shown in green solid line of FIG. 14(A), when the ceramics 11 temperature elevates by 65° C., the predicted flow saturated at depths of up to 3 cm (1.22 L/min), before sharply falling to 0.56 L/min at a depth of 4 cm. It can be seen that the nonlinearity of the relationship between temperature and blood flow leads to abrupt transitions between low, baseline flow and large, saturated flow. Indeed, the sharp sigmoidal transition (FIG. 7) predicts that large increases in blood flow are achievable at depths of 3 cm, where a flow of 1.03 L/min is predicted with the ceramic 11 temperature of 55° C. (as shown in yellow solid line in FIG. 14(A)).

As shown in FIG. 15(A), when considering blood flow as a function of lateral displacement from the center of the ceramics 11, it can be confirmed that, given a sufficiently high input temperature (e.g., 55° C.), the blood flow at a depth of 2 cm saturated across a large lateral extent of the lower back, and as shown in FIG. 15(B), even at a depth of 3 cm, the predicted flow produced by the ceramics 11 temperature of 55° C. reached a three-fold increase at a position in contact with the ceramics 11. Overall, the effective sensitivity of blood flow to distance from the heat source is less than that observed with temperature.

At this time, the step of calculating the thermal conduction and the temperature change due to the thermal effect when the user's body is pressed (Step 400) may include a step of visualizing the thermal conduction and the temperature change value of the user's body. The user's body may be any one or more parts of the user's body, such as the calculated skin, muscle, vertebrae, intervertebral disc.

In addition, the step of calculating the thermal conduction and the temperature change due to the thermal effect when the user's body is pressed (Step 400) may be a step of calculating the temperature change of a portion corresponding to a depth of 3 cm from the surface of the user's skin while the ceramics 11 of the spine thermal device 10 rises along the height direction Z and pressurizes the user's body. That is, since the portion corresponding to the depth of 3 cm from the skin surface is located with the user's spine and the muscle surrounding the spine (e.g., erector spinae muscle), blood circulation (blood flow improvement) of the portion may be maximized, and safety may be secured by checking in advance whether there is a burn risk for the user in a situation where the blood circulation of the portion corresponding to the depth of 3 cm is maximized.

In this case, the step of setting of the set value of the spinal thermal device 10 may be a step of setting the temperature of the ceramics 11, the ceramic height, and the heating element temperature, and may be changed according to the thermal massage mode selected by the user.

Meanwhile, after the step of visualizing the blood circulation value of the user's body (Step 600), the method may further include deriving the set value for obtaining an optimal temperature or an optimal blood circulation value of the user's body within a range of the ceramics 11 temperature, the ceramics 11 height, and the heating element temperature. That is, by visualizing the blood circulation value of the user's body, the set value is derived so that an optimal temperature or optimal blood circulation value suitable for the user can be obtained.

At this time, the step of deriving the set value for obtaining an optimal temperature or an optimal blood circulation value of the user's body may be a step of deriving the set value for obtaining the optimal temperature or the optimal blood circulation value for each user's body type according to a change in structure according to the user's body type. In other words, since thermal conduction and temperature changes vary depending on the BMI value, and at the same time, whether there is a risk of getting burned or not, it may be necessary to derive the optimal temperature and blood circulation value according to the BMI value, and when the optimum temperature and the blood circulation value are reflected in the spinal thermal device 10, optimal thermal massage for various users is possible, and precautions can be conveyed so that the user can avoid burns during the thermal massage process.

In this case, the step of converting the temperature change value of the user's body into the blood circulation value (Step 500) further comprises correcting the temperature change value of the user's body by reflecting blood circulation data for each user's body position. For example, the blood circulation data for each user's body position may be obtained through a laser Doppler imaging technique, and when the temperature change value of the user's body is corrected in this manner, a blood flow distribution may be more accurately derived, and thus it is possible to derive a set value of the spine thermal device 10 suitable for the user.

In addition, the step of correcting the temperature change value of the user's body of the user further comprises analyzing a laser received from the blood of the user, and for example, it may be configured to analyze the laser received from the user's blood by utilizing the above-described laser Doppler imaging technology, and correct the temperature change value of the user's body by reflecting the analysis result.

As described above, in the method for predicting the safety and effectiveness of the spinal thermal device according to an embodiment of the present disclosure, a blood flow improvement effect provided to a target site is optimized by generating three-dimensional structure data of the user's body and three-dimensional structure data of the spinal thermal device 10, calculating the thermal conduction and the temperature change of the user's body due to heat during the operation of the spinal thermal device 10 according to the set value, and then converting the calculated thermal conduction and the temperature change to the blood circulation value, and thus the safety and effectiveness of the spinal thermal device may be predicted.

Logical blocks, modules or units described in connection with embodiments disclosed herein can be implemented or performed by a computing device having at least one processor, at least one memory and at least one communication interface. The elements of a method, process, or algorithm described in connection with embodiments disclosed herein can be embodied directly in hardware, in a software module executed by at least one processor, or in a combination of the two. Computer-executable instructions for implementing a method, process, or algorithm described in connection with embodiments disclosed herein can be stored in a non-transitory computer readable storage medium.

Although the present disclosure has been described above, the spirit of the present disclosure is not limited by the embodiments presented in the specification, and those skilled in the art who understand the spirit of the present disclosure may easily propose other embodiments by adding, modifying, deleting, adding, etc., elements within the same spirit, but the spirit of the present disclosure is also given.

| | |
|---|---|
| 10 spinal thermal device | 11 ceramic |
| 12 driving unit | 13 support unit |
| 13a first support member | 13b second support member |
| X length direction | Y width direction |
| Z height direction | |

What is claimed is:

1. A method for predicting safety and effectiveness of a spinal thermal device using a computer, the method comprising:
generating three-dimensional structure data of a user's body;
generating three-dimensional structure data of the spinal thermal device;
setting a set value of the spinal thermal device;
calculating a thermal conduction and a temperature change of the user's body due to heat applied in a process of pressurizing the user's body as the spinal thermal device operates as the set value;
converting the temperature change value of the user's body into a blood circulation value; and
visualizing the blood circulation value of the user's body.

2. The method of claim 1, wherein calculating the thermal conduction and the temperature change of the user's body due to heat applied in a process of pressurizing the user's body comprises visualizing the thermal conduction and the temperature change value of the user's body.

3. The method of claim 1, wherein calculating the thermal conduction and the temperature change of the user's body due to heat applied in a process of pressurizing the user's body comprises calculating the temperature change of a portion corresponding to a depth of 3 cm from a skin surface of the user in a process of pressurizing the user's body while a ceramic of the spinal thermal device rises along a height direction.

4. The method of claim 1, wherein calculating the thermal conduction and the temperature change of the user's body comprises calculating the thermal conduction and the temperature change through Equation 1 below:
$$ρ c \frac{\partial T}{\partial t} = ∇ \cdot ( k ∇ T ) + {ρ}_{b} {c}_{b} {w}_{b} ( T - {T}_{b} ) + {Q}_{m} \qquad ( 1 )$$
where ρ is tissue density (units of kg/m³), c is specific heat capacity (J kg⁻¹ K⁻¹), T is temperature (K), t denotes time (s), k is thermal conductivity (W m⁻¹ K⁻¹), ρ_{b} is density of blood (kg m⁻³), c_{b} is specific heat capacity of blood (J kg⁻¹ K⁻¹), w_{b} is perfusion rate of blood (1/s), T_{b} is blood temperature (K), Qₘ is rate of metabolic heat generation (W/m³), and solution is carried out under steady state conditions.

5. The method of claim 4, wherein calculating through the Equation 1 further comprises calculating a convective heat loss from boundaries of the user's body to external environment through Equation 2 below:
q₀=h(T_{amb}−T) (2)
where q₀ (W m⁻²) is convective heat flux, h is heating transfer coefficient for the tissues comprising the model (e.g., user's body), and is 5 W m⁻² K, T_{amb} is ambient temperature (e.g., 25° C.), initial temperature of the tissues is set to 37° C., temperature of the ceramic is fixed within a simulation to a value ranging from 45 to 65° C., and external boundary of a skin in contact with a bed surface, corresponding to a heating mat, is assigned a temperature of 40° C.

6. The method of claim 1, wherein converting the temperature change value of the user's body into the blood circulation value comprises converting the temperature change value of the user's body into a blood circulation value through Equation 3 below:
F(ΔT)=Fₒ+(Fₘₐₓ−Fₒ)e^{-(aΔT+b)} (3)
where F is blood flow (L/min), ΔT is change in temperature at the site of vessel (° C.), Fₒ is blood flow in absence of exogenous heating, Fₘₐₓ is maximum achievable blood flow due to physical limitations of cardiovascular system, and the free model parameters a and b are estimated by fitting model (e.g., the user's body) to empirical measurements reported by Chiesa and colleagues.

7. The method of claim 1, wherein setting the set value of the spinal thermal device comprises setting a ceramic temperature, a ceramic height, and a heating element temperature.

8. The method of claim 1, further comprising after visualizing of the blood circulation value of the user's body, deriving a set value for obtaining an optimal temperature or an optimal blood circulation value of the user's body within a range of a set ceramic temperature, a ceramic height, and a heating element temperature.

9. The method of claim 8, wherein deriving the set value for obtaining an optimal temperature or an optimal blood circulation value of the user's body comprises deriving the set value for obtaining the optimal temperature or the optimal blood circulation value for each user's body type according to a change in structure according to the user's body type.

10. The method of claim 1, wherein converting the temperature change value of the user's body into the blood circulation value further comprises correcting the temperature change value of the user's body by reflecting blood circulation data for each user's body position.

11. The method of claim 10, wherein correcting the temperature change value of the user's body further comprises analyzing a laser received from the blood of the user.

12. The method of claim 1, wherein the three-dimensional structure data of the user's body is three-dimensional structure data classified into skin, subcutaneous fat, soft tissue, muscles, vertebrae, intervertebral disc, epidural fat, cerebrospinal fluid, and spinal cord.
